(19) United States
(12) Patent Application Publication
(10) Pub. No.: US 2024/0415516 A1
(43) Pub. Date: Dec. 19, 2024

(54) THRU THE SCOPE TISSUE CLOSURE DEVICE

(71) Applicant: Boston Scientific Scimed, Inc., Maple Grove, MN (US)

(72) Inventors: Gonzalo Jose Saenz Villalobos, Heredia (CR); Carolina Padilla Castro, San Jose (CR); Oscar Segura Elizondo, San Jose (CR); Gabriela Gamboa Bastos, San Jose (CR)

(73) Assignee: Boston Scientific Scimed, Inc., Maple Grove, MN (US)

(21) Appl. No.: 18/742,038

(22) Filed: Jun. 13, 2024

Related U.S. Application Data
(60) Provisional application No. 63/508,048, filed on Jun. 14, 2023.

(51) Int. Cl. A61B 17/122; A61B 17/00; A61B 17/12

(52) U.S. Cl. CPC A61B 17/122; A61B 2017/00367; A61B 2017/12004

(57) ABSTRACT

An example medical device is disclosed. The example medical device includes a tissue clipping device including a bushing having a distal end, a proximal end, and a lumen extending therethrough. A clip including at least a first arm and a second arm is configured to be coupled to the distal end of the bushing, wherein one of the first arm and the second arm includes a tissue grasping member. A control wire having a proximal end and a distal end, is configured to extend through the lumen of the bushing, and a helical member is configured to be coupled to the distal end of the control wire. The control wire is configured to advance the helical member in a distal direction along a longitudinal axis of the tissue clipping device, and retract the helical member in a proximal direction along the longitudinal axis of the tissue clipping device.

### CROSS-REFERENCE TO RELATED APPLICATIONS

This application claims the benefit of U.S. Provisional Patent Application Ser. No. 63/508,048 filed on Jun. 14, 2023, the disclosure of which is incorporated herein by reference.

### TECHNICAL FIELD

The present disclosure pertains to medical devices, and methods for manufacturing medical devices. More particularly, the present disclosure pertains to a full thickness tissue closure device.

### BACKGROUND

Pathologies of the gastrointestinal system, the biliary tree, the vascular system, and other body lumens and hollow organs are often treated through endoscopic procedures, many of which require active and/or prophylactic hemostasis to control bleeding. Tools for deploying hemostatic clips via an endoscope are often used to stop internal bleeding by clamping together the edges of the wounds or incisions. Hemostasis clips grasp tissue surrounding a wound and hold edges of the wound together by applying pressure to the site to allow natural healing processes to close the wound. Specialized endoscopic clipping devices are used to deliver the clips to the desired locations within the body and to position and deploy the clips at the desired locations after which the clip delivery device is withdrawn, leaving the clip within the body. These clips may be left in place until they are sloughed off through natural processes or removed later through a separate procedure after the bleeding site has healed.

### BRIEF SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

Some embodiments can be implemented as a tissue closure device. The tissue closure device can comprise a bushing having a distal end, a proximal end, and a lumen extending therethrough; a clip including at least a first arm and a second arm, wherein a proximal end region of the clip is configured to be coupled to the distal end of the bushing; a control wire having a proximal end and a distal end, the control wire configured to extend through the lumen of the bushing; and a helical member configured to be coupled to the distal end of the control wire; wherein the control wire is configured to advance the helical member in a distal direction along a longitudinal axis of the tissue closure device, and retract the helical member in a proximal direction along the longitudinal axis of the tissue closure device; and wherein one of the first arm and the second arm includes a tissue grasping member.

With further embodiments of the tissue clipping device, the tissue grasping member is a hook.

With further embodiments of the tissue clipping device, the helical member is coupled to the control wire via a cam member.

With further embodiments of the tissue clipping device, when the control wire is retracted in the proximal direction, the cam member engages with a proximal end of the first arm and a proximal end of the second arm, thereby causing the first arm and the second arm to move toward one another to a closed position.

With further embodiments of the tissue clipping device, when the control wire is retracted proximally the cam member is positioned within a proximal end of the clip thereby locking the first arm and the second arm in the closed position.

With further embodiments of the tissue clipping device, the proximal end of the control wire is coupled to a handle.

With further embodiments of the tissue clipping device, when the clip is in an open position, a proximal end of the first arm and a proximal end of the second arm engage with one another to control an opening angle.

Some embodiments of the disclosure can be implemented as a method to closure tissue with a tissue closure device. The method can comprise inserting a closure device through a natural body lumen to a target site within a body, the closure device comprising: a bushing having a distal end, a proximal end, and a lumen extending therethrough; a clip including at least a first arm and a second arm, wherein a proximal end of the clip is configured to be coupled to the distal end of the bushing; a control wire having a proximal end and a distal end, the control wire configured to extend through the lumen of the bushing; and a helical member configured to be coupled to the distal end of the control wire; advancing the control wire distally so that the first arm and the second arm move away from one another, radially with respect to a longitudinal axis of the clipping device, and the helical member engages a first tissue at the target site; rotating the control wire in a first direction, which rotates the helical member into the tissue; and retracting the control wire and the helical member in a proximal direction thereby drawing the first tissue proximally into the clip; wherein when the control wire and helical member are retracted proximally, the first arm and the second arm move toward one another, thereby closing the clip.

In further embodiments of the method, one of the first arm and the second arm includes a tissue grasping member.

In further embodiments of the method, the tissue grasping member is a hook.

In further embodiments of the method, the helical member is coupled to the control wire via a cam member.

In further embodiments of the method, when the control wire is retracted proximally, the cam member engages with a proximal end of the first arm and a proximal end of the second arm, thereby causing the first arm and the second arm to move toward one another to close the clip.

In further embodiments, the method can comprise rotating the control wire in a second direction, which rotates the helical member, thereby disengaging the helical member from the first tissue.

In further embodiments of the method, when the control wire is retracted proximally the cam member is positioned within a proximal end of the clip thereby locking the first arm and the second arm in a closed position.

In further embodiments, the method can comprise drawing the control wire proximally to sever a connection between the control wire and the clip, thereby permitting the control wire to be withdrawn from the tissue clipping device.

In further embodiments, the method can comprise advancing the control wire so that the first arm and the second arm move away from one another, radially with respect to a longitudinal axis of the clipping device, and the helical member engages a second tissue at the target site.

In further embodiments of the method, when the helical member engages the second tissue, the first tissue is secured to a tissue grasping member.

Some embodiments of the disclosure can be implemented as a tissue clip. The tissue clip can comprise a bushing having a distal end and a proximal end; a first arm including a first tissue grasping member positioned adjacent a distal end of the first arm, wherein a proximal end of the first arm coupled to the distal end of the bushing; a second arm including a second tissue grasping member positioned adjacent a distal end of the second arm, wherein a proximal end of the second arm coupled to the distal end of the bushing; and a helical member configured to be positioned between the first arm and the second arm, wherein the helical member is configured to advance distally along a longitudinal axis of the tissue clip; wherein the tissue clip is configured to move from a first, closed position to a second, open position.

In further embodiments of the tissue clip, when the clip is in the second, open position, a proximal end of the first arm and a proximal end of the second arm engage with one another to control an opening angle.

In further embodiments of the tissue clip, one of the first tissue grasping member or the second tissue grasping member includes a hook.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates an example tissue closure device in a first position;

FIG. 2 illustrates the tissue closure device shown in FIG. 1 in a second position;

FIG. 3 illustrates a portion of the tissue closure device shown in FIG. 1;

FIG. 4 illustrates an example tissue closure device positioned near a target site;

FIG. 5A illustrates an enlarged portion of the tissue closure device showing a helical member, shown in Circle 5A in FIG. 4;

FIG. 5B illustrates the helical member shown in FIG. 5A engaged with a target tissue;

FIG. 5C illustrates the tissue closure device shown in FIG. 4 wherein the helical member is retracted proximally;

FIG. 5D illustrates a cross-sectional view of the tissue closure device shown in FIG. 5C including the target tissue, wherein the tissue closure device is in a first position;

FIG. 5E illustrates the tissue closure device shown in FIG. 5D including the target tissue, wherein the tissue closure device is in a second position;

FIG. 5F illustrates the tissue closure device shown in FIG. 5E including the helical member engaged with a target tissue, wherein the tissue closure device is in the first position;

FIG. 5G illustrates the tissue closure device shown in FIG. 5F including the target tissue, wherein the tissue closure device is in the second position;

FIG. 5H illustrates a cross-sectional view of a portion of the tissue closure device shown in FIG. 5G;

FIG. 5I illustrates the portion of the tissue closure device shown in FIG. 5H wherein a control wire has been removed;

FIG. 6 illustrates an example tissue closure device attached to a target site;

FIG. 7A illustrates a cross-sectional view of a portion of an example tissue closure device in a first position;

FIG. 7B illustrates the cross-sectional view of the portion of the example tissue closure device shown in FIG. 7A in a second position;

FIG. 8A illustrates a cross-sectional view of a portion of an example tissue closure device in a first position;

FIG. 8B illustrates the cross-sectional view of the portion of the example tissue closure device shown in FIG. 8A in a second position;

FIG. 9A illustrates a cross-sectional view of a portion of an example tissue closure device in a first position;

FIG. 9B illustrates the cross-sectional view of the portion of the example tissue closure device shown in FIG. 9A in a second position;

FIG. 10 illustrates an example tissue closure device;

FIG. 11A illustrates an example tissue closure device in a first position; and

FIG. 11B illustrates the example tissue closure device shown in FIG. 11A in a second position.

### DETAILED DESCRIPTION

While the disclosure is amenable to various modifications and alternative forms, specifics thereof have been shown by way of example in the drawings and will be described in detail. It should be understood, however, that the intention is not to limit the disclosure to the embodiments described. On the contrary, the intention is to cover all modifications, equivalents, and alternatives falling within the spirit of the disclosure.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes, 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in this specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the claims.

Pathologies of the body lumens and hollow organs are often treated through endoscopic procedures, many of which may require mechanisms to control bleeding. Tools for deploying tissue closure devices, such as hemostatic clips, via an endoscope are often used to stop internal bleeding by clamping together the edges of the wounds or incisions. Hemostasis clips (e.g., wound/tissue closure devices) may grasp tissue surrounding a wound and hold the edges of the wound together by applying pressure to the target tissue site to allow natural healing processes to close the wound. Specialized endoscopic clipping devices are used to deliver the clips to the desired locations within the body and to position and deploy the clips at the desired locations after which the clip delivery device is withdrawn, leaving the clip within the body. These clips may be left in place until they are removed via natural processes or later through a separate procedure after the bleeding site has healed.

As those skilled in the art will understand, when deployed to tissue surrounding a bleeding wound, the clip arms draw the edges of the wound together effecting hemostasis of the target tissue site. However, as those skilled in the art will understand, these clips may be used for any application in which portions of tissue need to be drawn together. As the control wire is pushed distally, the clip holder releases its grasp on the proximal end of the clip, deploying the clip from an insertion device. The insertion device is then removed from the body, leaving the clip in place over the target tissue. The exemplary clip according to the invention may be used for fastening tissue layers together, for closing an opening in one or more layers of tissue, for lung tissue compression, to compress bronchiole/alveoli tissue in emphysema patients, for the treatment of Chronic Obstructive Pulmonary Disease (“COPD”), etc. For example, the clipping device may be used to close wounds and/or incisions for hemostasis of natural or surgical bleeding, “stitching” a wound, occluding a vessel or lumen, plicating a hollow organ, attaching tissues, tissue approximation, etc.

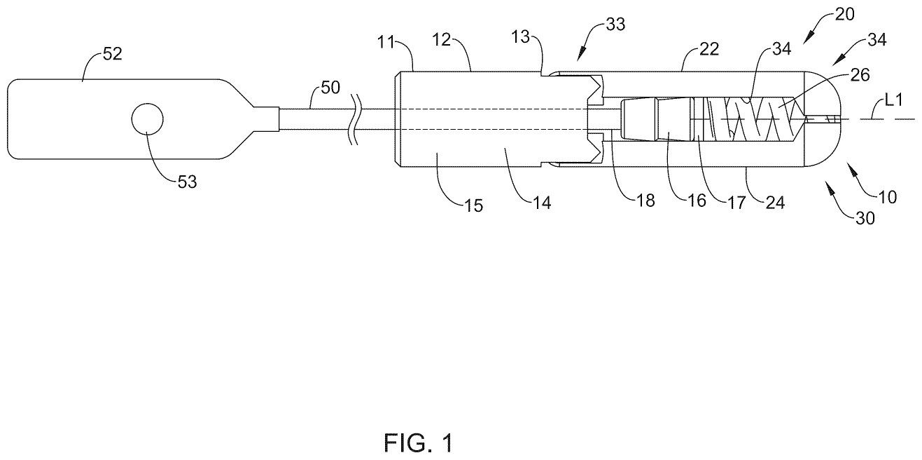

FIGS. 1 to 3 illustrate an example tissue closure device 10. FIG. 1 illustrates the example tissue closure device 10 in a first, closed position 30. The tissue closure device 10 may include a bushing 12, which may include a distal end 13 and a proximal end 11. The bushing 12 may include a lumen 15 extending therethrough. In some cases, the proximal end 11 of the bushing 12 may be configured to be coupled to a shaft 50. In some cases, the shaft 50 may include an endoscope, a laparoscope, a catheter, a guide tube, or the like. The distal end 13 of the bushing 12 may be releasably coupled to a proximal end region 33 of a clip 20. The clip 20 may include a diameter that may allow the clip 20 and the bushing 12 to be passed through a delivery device. The clip 20 may include at least a first arm 22, a second arm 24. While it is shown that the clip 20 includes the first arm 22 and the second arm 24, it may be contemplated that the clip 20 may include three arms, four arms, five arms, or any suitable number of arms as desired. The first arm 22 and the second arm 24 may be spaced equidistantly from one another, which may position the first arm 22 and the second arm 24 180 degrees from one another, although other angles may be used without deviating from the scope of the disclosure. The first arm 22 and the second arm 24 may be formed with a curvature selected such that, when the first arm 22 and the second arm 24 are in the first, closed position 30, as shown in FIG. 1, a tissue-receiving cavity 34 may be formed between the first arm 22 and the second arm 24.

The clip 20 may further include a helical member 26 that may be positioned between the first arm 22 and the second arm 24 within the tissue-receiving cavity 34. In some cases, the helical member 26 may be coupled to a washer 17, which may be coupled to a cam member 16. In some cases, the washer 17 may be removed, and the helical member 26 may be coupled directly to the cam member 16. The helical member 26 may be releasably coupled to a distal end 18 of the control wire 14. In some cases, the control wire 14 may be releasably coupled to the cam member 16. In some cases, the control wire 14 may be releasably coupled to the washer 17. The control wire 14 may extend through a lumen of the shaft 50 and the lumen 15 of the bushing 12, and extend beyond the distal end 13 of the bushing 12. The control wire 14 may be configured to be actuated along a longitudinal axis L₁ of the tissue closure device 10. Actuation of the control wire 14 may include advancing the control wire 14 in a distal direction and/or retracting the control wire 14 in a proximal direction. Further, the control wire 14 may be configured to be rotated clockwise and/or counter-clockwise. Actuation and/or rotation of the control wire 14 may then result in actuation and/or rotation of the helical member 26, as the helical member 26 is releasably coupled to the control wire 14.

FIG. 1 further illustrates that a proximal end 19 of the control wire 14 may be coupled to a control member 52 (e.g., a handle, actuator, etc.). The control member 52 may be configured to control the actuation of the control wire 14 (e.g. advancing, retracting, rotating the control wire 14). In some cases, the control member 52 may further be utilized as a grip to control the translation of the shaft 50. The control member 52 may be utilized by a clinician to advance the tissue closure device 10 to a position adjacent a target tissue to perform a medical treatment. Additionally, the control member 52 may include one or more actuators, gears, levers, etc. which allow a clinician to manipulate the shaft 50 in addition to other features or components, such as the control wire 14, of the tissue closure device 10. As will be described in greater detail below, the tissue closure device 10 may be advanced distally within a portion of a body lumen to a position adjacent a target tissue, such as a lesion, while the shaft 50 may extend out of the body lumen to a position outside of the body.

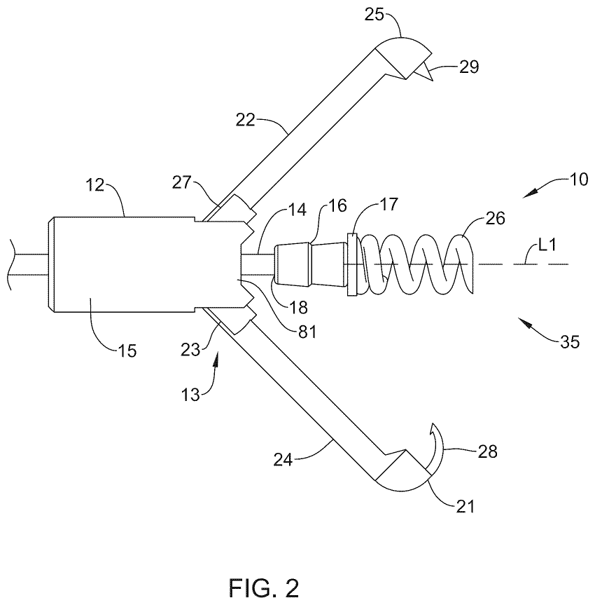

FIG. 2 illustrates the tissue closure device 10 in a second, open position 35. As shown in FIG. 2, a proximal end 27 of the first arm 22 may include a first tab 31 which may include a size and shape configured to engage with a first cut-out 81 within the distal end 13 of the bushing 12. The second arm 24 may include a second tab 32 which may include a size and shape configured to engage with a second cut-out 82 within the distal end 13 of the bushing 12. The cut-outs 81, 82 may be sized to allow for the first and second tabs 31, 32 to move within the cut-outs 81, 82 as the first arm 22 and the second arm 24 radially expand away from the longitudinal axis Lj of the tissue closure device 10. When the first arm 22 and the second arm 24 radially expand to the second, open position 35, the first tab 31 and the second tab 32 abut one another, thereby preventing the first arm 22 and the second arm 24 from further expansion and control an opening angle of the clip 20. In some cases, full expansion of the first arm 22 and the second arm 24 may include a range of about 25 degrees to about 90 degrees from the longitudinal axis L₁ of the tissue closure device 10. In some cases, full expansion of the first arm 22 and the second arm 24 may include a range of about 35 degrees to about 60 degrees from the longitudinal axis L₁ of the tissue closure device 10. In some cases, full expansion of the first arm 22 and the second arm 24 may include 45 degrees from the longitudinal axis L₁ of the tissue closure device 10.

As shown in FIG. 2, the first arm 22 and the second arm 24 may include blunt ends or rounded ends to be less traumatic to tissue. In some cases, a distal end 25 of the first arm 22 may include a first tissue grasping member 29, and a distal end 21 of the second arm 24 may include a second tissue grasping member 28. In some cases, the first tissue grasping member 29 may include a pyramid point. In some cases, the second tissue grasping member 28 may include a hook. While it is illustrated that the first tissue grasping member 29 includes a pyramid point and the second tissue grasping member 28 includes a hook, it may be contemplated that the first tissue grasping member 29 and/or the second tissue grasping member 28 may include a pyramid point, a hook, a plurality of teeth-like shaped ends, a barb, or the like. In some cases, one of the first arm 22 or the second arm 24 may include a recess within the distal end configured to receive a tissue grasping member (e.g., the first tissue grasping member 29, the second tissue grasping member 28). These are some examples.

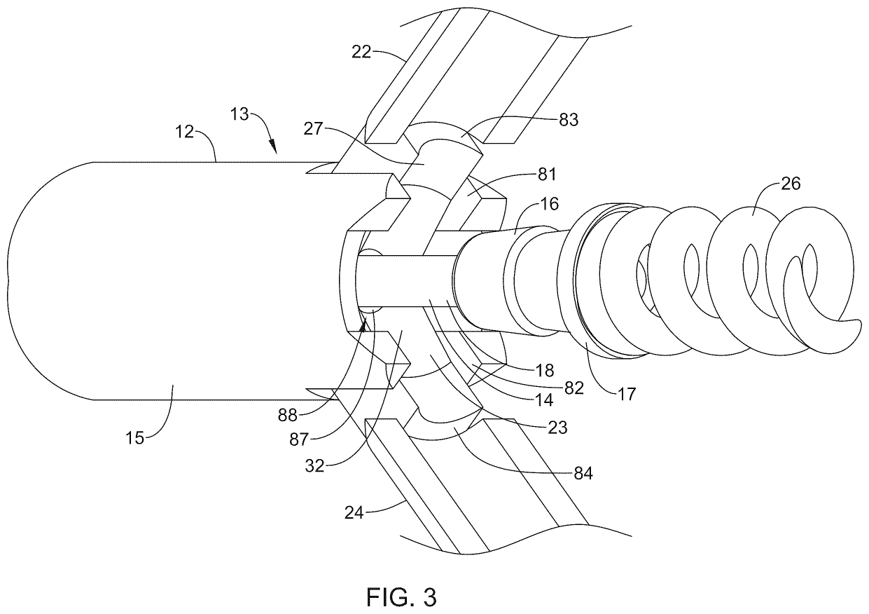

FIG. 3 illustrates a portion of the tissue closure device 10 showing the distal end 13 of the bushing 12 and the proximal ends 27, 23 of the first arm 22 and the second arm 24, respectively. As shown in FIG. 3, the tissue closure device 10 is in the second, open position 35. When the tissue closure device 10 is in the second, open position 35, the first tab 31 and the second tab 32 abut one another at outer edges of the first tab 31 and the second tab 32. The first tab 31 may include a first notch 86 and the second tab 32 may include a second notch 87 such that when the first tab 31 and the second tab 32 abut one another, they form an opening 88. The opening 88 includes a size and shape such that the control wire 14 may be actuated freely within the tissue closure device 10.

In some cases, the control wire 14 may be retracted proximally thereby allowing the cam member 16 to engage with (e.g. abut) the first tab 31 and the second tab 32. Proximal retraction of the control wire 14 may provide a compressive force of the cam member 16 onto the first tab 31 and the second tab 32, thereby causing the first arm 22 and the second arm 24 to radially compress toward the longitudinal axis L1 of the tissue closure device 10. This compressive force on the tabs 31, 32 thereby moves the clip 20 from the second, open position 35 to the first, closed position 30. Further, the proximal end 27 of the first arm 22 may include a first ledge 83 and the proximal end 23 of the second arm 24 may include a second ledge 84 each positioned on an inside of each arm 22, 24. The ledges 83, 84 may be configured to receive the cam member 16 and hold the cam member 16 in place when the control wire 14 is removed from the clip 20, thereby locking the first arm 22 and the second arm 24 in the first, closed position 30, and preventing the cam member 16 from being removed from the clip 20. Removal of the control wire 14 may include drawing the control wire 14 proximally to sever a connection between the control wire 14 and the clip 20 (e.g., the cam member 16), which permits the control wire 14 to be withdrawn from the tissue closure device 10.

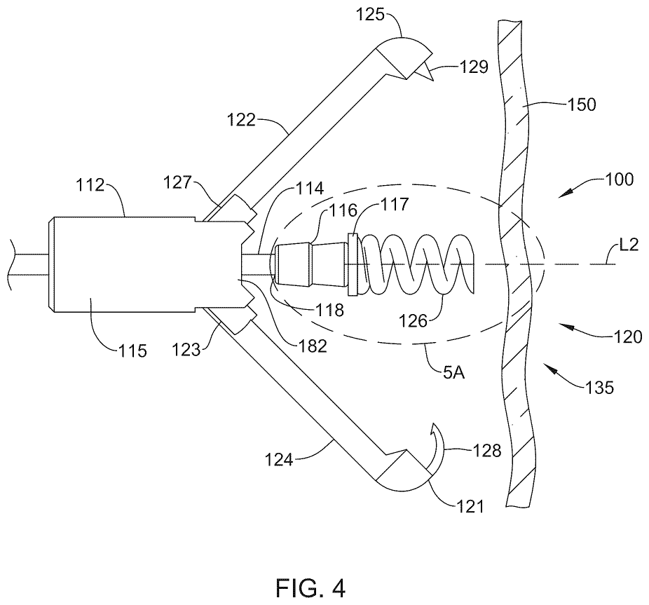

FIG. 4 illustrates an example tissue closure device 100 positioned near a target tissue site 150. The tissue closure device 100 may be considered to be an example of the tissue closure device 10 shown in FIGS. 1 to 3. The tissue closure device 100 may include a bushing 112, which may include a distal end 113 and a proximal end 111. The bushing 112 may include a lumen 115 extending therethrough. In some cases, the proximal end 111 of the bushing 112 may be configured to be coupled to a shaft (e.g., shaft 50). In some cases, the shaft may include an endoscope, a laparoscope, a catheter, a guide tube, or the like. The distal end 113 of the bushing 1112 may be releasably coupled to a proximal end region 133 of a clip 120. The clip 120 may include a diameter that may allow the clip 120 and the bushing 112 to be passed through a delivery device. The clip 120 may include at least a first arm 122, a second arm 124. While it is shown that the clip 120 includes the first arm 122 and the second arm 124, it may be contemplated that the clip 120 may include three arms, four arms, five arms, or any suitable number of arms as desired. The first arm 122 and the second arm 124 may be spaced equidistantly from one another, which may position the first arm 122 and the second arm 124 180 degrees from one another, although other angles may be used without deviating from the scope of the disclosure. The first arm 122 and the second arm 124 may be formed with a curvature selected such that, when the first arm 122 and the second arm 124 are in a closed position (e.g., first, closed position 30), a tissue-receiving cavity 134 may be formed between the first arm 122 and the second arm 124.

The clip 120 may further include a helical member 126 that may be positioned between the first arm 122 and the second arm 124 within the tissue-receiving cavity 134. In some cases, the helical member 126 may be coupled to a washer 117, which may be coupled to a cam member 116. In some cases, the washer 117 may be removed, and the helical member 126 may be coupled directly to the cam member 116. The helical member 126 may be releasably coupled to a distal end 118 of the control wire 114. In some cases, the control wire 114 may be releasably coupled to the cam member 116. In some cases, the control wire 114 may be releasably coupled to the washer 117. The control wire 114 may extend through a lumen of the shaft and the lumen 115 of the bushing 112, and extend beyond the distal end 113 of the bushing 112. The control wire 114 may be configured to be actuated along a longitudinal axis L₂ of the tissue closure device 100. Actuation of the control wire 114 may include advancing the control wire 114 in a distal direction and/or retracting the control wire 114 in a proximal direction. Further, the control wire 114 may be configured to be rotated clockwise and/or counter-clockwise. Actuation and/or rotation of the control wire 114 may then result in actuation and/or rotation of the helical member 126, as the helical member 126 is releasably coupled to the control wire 114.

FIG. 4 illustrates the tissue closure device 100 in the second, open position 35. As shown in FIG. 4, a proximal end 127 of the first arm 122 may include a first tab 131 which may include a size and shape configured to engage with a first cut-out 181 within the distal end 113 of the bushing 112. The second arm 124 may include a second tab 132 which may include a size and shape configured to engage with a second cut-out 182 within the distal end 113 of the bushing 112. The cut-outs 181, 182 may be sized to allow for the first and second tabs 131, 132 to move within the cut-outs 181, 182 as the first arm 122 and the second arm 124 radially expand away from the longitudinal axis L₂ of the tissue closure device 100. When the first arm 122 and the second arm 124 radially expand to the open position 135, the first tab 131 and the second tab 132 abut one another, thereby preventing the first arm 122 and the second arm 124 from further expansion. In some cases, full expansion of the first arm 122 and the second arm 124 may include a range of about 25 degrees to about 90 degrees from the longitudinal axis L₂ of the tissue closure device 100. In some cases, full expansion of the first arm 122 and the second arm 124 may include a range of about 35 degrees to about 60 degrees from the longitudinal axis L₂ of the tissue closure device 100. In some cases, full expansion of the first arm 122 and the second arm 124 may include 45 degrees from the longitudinal axis L₂ of the tissue closure device 100.

As shown in FIG. 4, the first arm 122 and the second arm 124 may include blunt ends or rounded ends to be less traumatic to tissue. In some cases, a distal end 125 of the first arm 122 may include a first tissue grasping member 129, and a distal end 121 of the second arm 124 may include a second tissue grasping member 128. In some cases, the first tissue grasping member 129 may include a pyramid point. In some cases, the second tissue grasping member 128 may include a hook. While it is illustrated that the first tissue grasping member 129 includes a pyramid point and the second tissue grasping member 128 includes a hook, it may be contemplated that the first tissue grasping member 129 and/or the second tissue grasping member 128 may include a pyramid point, a hook, a plurality of teeth-like shaped ends, a barb, or the like. In some cases, one of the first arm 122 or the second arm 124 may include a recess within the distal end configured to receive a tissue grasping member (e.g., the first tissue grasping member 129, the second tissue grasping member 128). These are some examples.

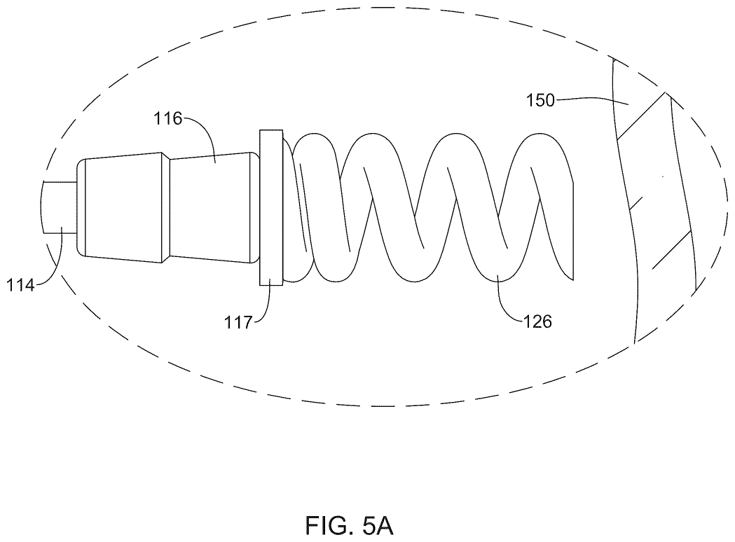

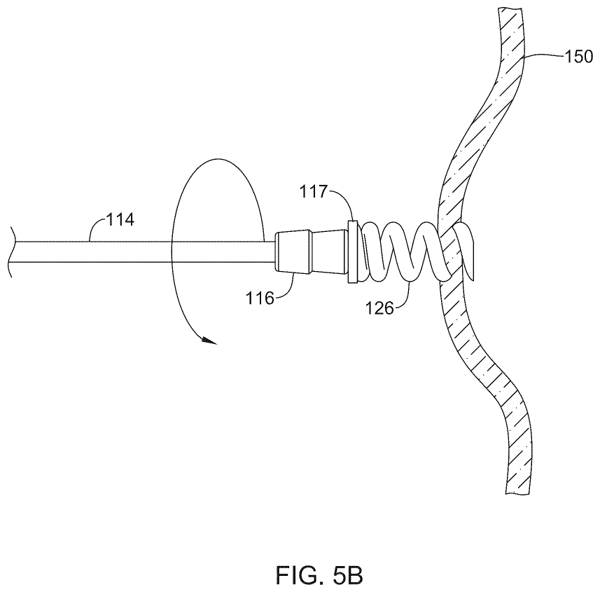

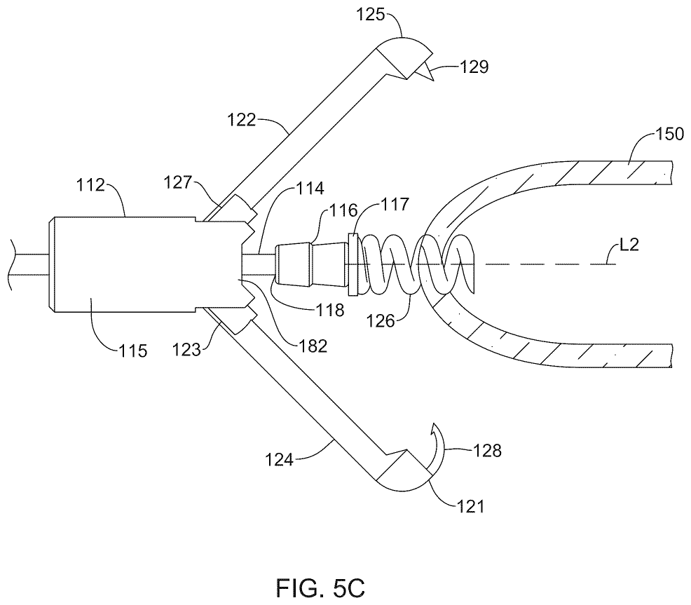

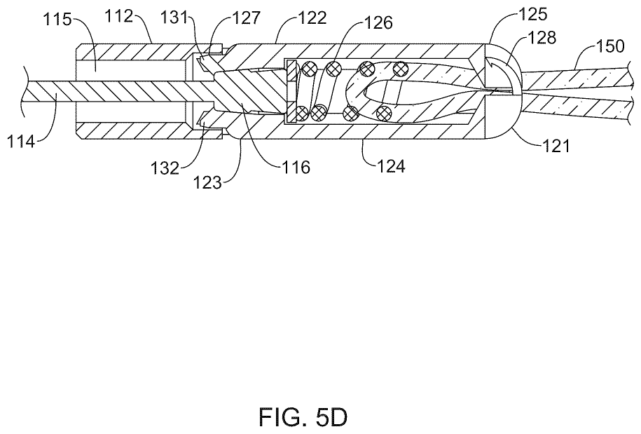

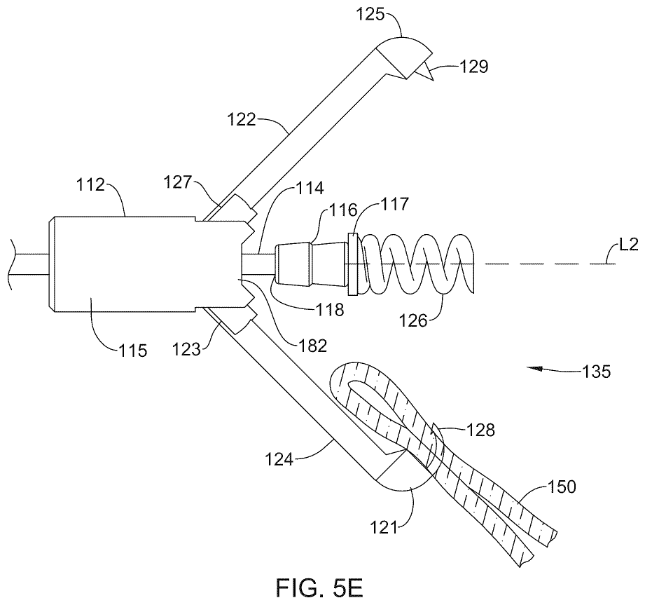

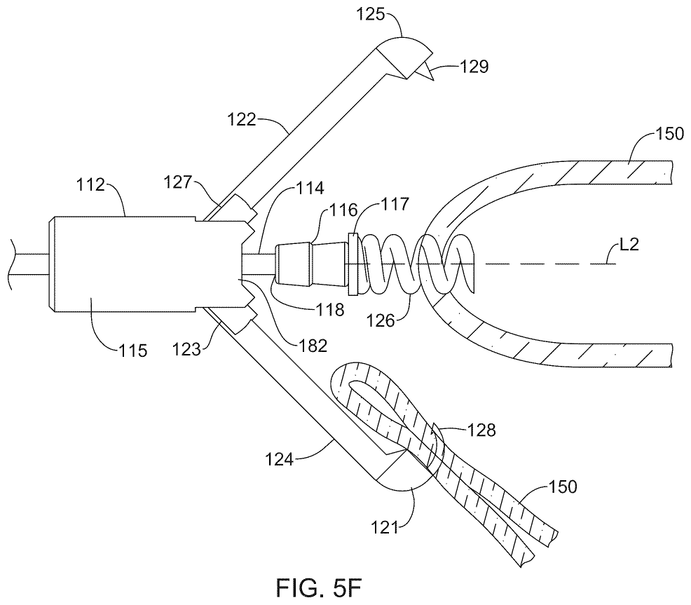

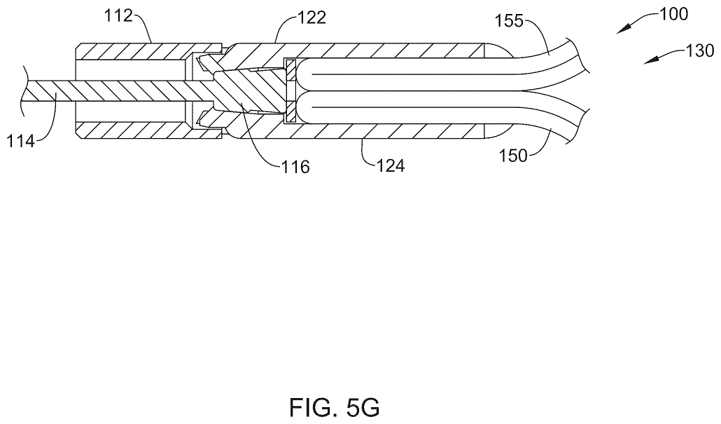

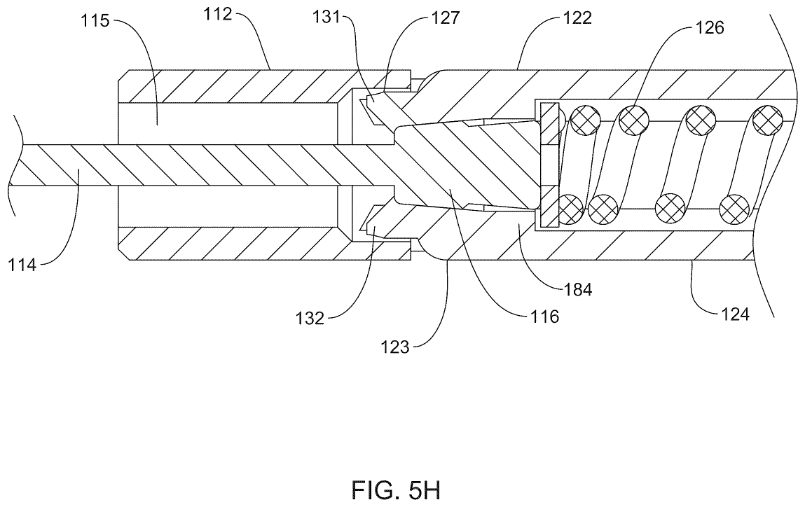

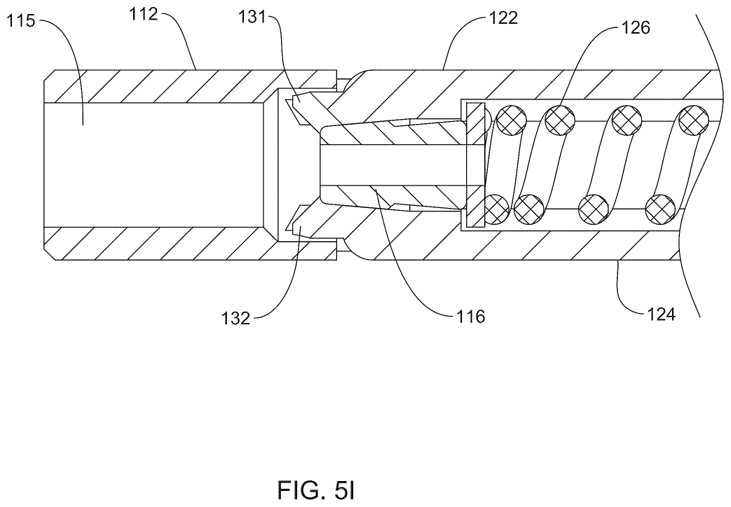

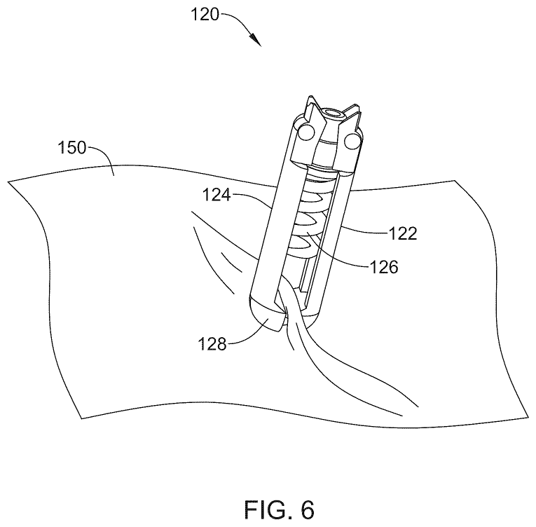

As shown in FIG. 4, the tissue closure device 100 is positioned adjacent the target tissue site 150. The control wire 114 may be advanced distally along the longitudinal axis L₂ of the tissue closure device 100, thereby advancing the helical member 126 toward the target tissue site 150. The control wire 114 may advance the helical member 126 until it abuts the target tissue site 150 and a user may rotate the control wire 114 in a first direction, which rotates the helical member 126 into the target tissue site 150, as will be shown further in FIGS. 5A to 6.

FIGS. 5A to 6 illustrate a method of tissue closure in accordance with the disclosure. FIG. 5A shows the helical member 126 positioned near the target tissue site 150, as in Circle 5A of FIG. 4. As previously stated, the control wire 114 may be advanced distally along the longitudinal axis L₂ of the tissue closure device 100, thereby advancing the helical member 126 toward the target tissue site 150. The control wire 114 may advance the helical member 126 until it abuts the target tissue site 150 and a user may rotate the control wire 114 in a first direction, as indicated by arrow 190, which rotates the helical member 126 into the target tissue site 150, as shown in FIG. 5B. The control wire 114 may then be retracted proximally along the longitudinal axis L₂ of the tissue closure device 100 until the cam member 116 to engages with (e.g. abuts) the first tab 131 and the second tab 132, as shown in FIG. 5C. Proximal retraction of the control wire 114 may provide a compressive force of the cam member 116 onto the first tab 131 and the second tab 132, thereby causing the first arm 122 and the second arm 124 to radially compress toward the longitudinal axis L₂ of the tissue closure device 100. This compressive force on the tabs 131, 132 thereby moves the clip 120 from the open position 135 to a closed position 130, as shown in FIG. 5D.

When the clip 120 is in the closed position 130, the first tissue grasping member 129 may push the target tissue site 150 onto the second tissue grasping member 128, as shown in FIG. 5D. The second tissue grasping member 128 may include a “fish hook” type hook that may be configured to hold the tissue onto the second arm 124. The control wire 114 may then be rotated in a second direction, as indicated by arrow 191, which rotates the helical member 126, thereby disengaging the helical member 126 from the target tissue site 150. The control wire 114 may then be advanced distally, thereby advancing the helical member 126 distally. When the control wire 114 and helical member 126 advance in a distal direction, the first arm 122 and the second arm 124 are biased to move from the closed position 130 to an open position 135, as shown in FIG. 5E.

As shown in FIG. 5E, the second tissue grasping member 128 may continue to grasp the tissue of the target tissue site 150. This allows the helical member 126 to advance into a second target tissue site 155, as shown in FIG. 5F, and grasp more tissue. The control wire 114 may then be retracted proximally along the longitudinal axis L₂ of the tissue closure device 100 until the cam member 116 to engages with (e.g. abuts) the first tab 131 and the second tab 132. Proximal retraction of the control wire 114 may provide a compressive force of the cam member 116 onto the first tab 131 and the second tab 132, thereby causing the first arm 122 and the second arm 124 to radially compress toward the longitudinal axis L₂ of the tissue closure device 100. This compressive force on the tabs 131, 132 thereby moves the clip 120 from the open position 135 to a closed position 130. When the clip 120 is in the closed position 130, the second target tissue site 155 may be gripped by the second tissue grasping member 128, as shown in FIG. 5G. The control wire 114 may then be rotated in a second direction, which rotates the helical member 126, thereby disengaging the helical member 126 from the second target tissue site 155. This process of grasping target tissue may be repeated as often as the clinician desires.

As shown in FIG. 5H, the proximal end 127 of the first arm 122 may include a first ledge 183 and the proximal end 123 of the second arm 124 may include a second ledge 184 each positioned on an inside of each arm 122, 124. The ledges 183, 184 may be configured to receive the cam member 116 and hold the cam member 116 in place when the control wire 114 is removed from the clip 120, thereby locking the first arm 122 and the second arm 124 in the closed position 130, and preventing the cam member 116 from being removed from the clip 120. Removal of the control wire 114 may include drawing the control wire 114 proximally to sever a connection between the control wire 114 and the clip 120 (e.g., the cam member 116), which permits the control wire 114 to be withdrawn from the tissue closure device 100, as shown in FIG. 5I. The tissue clip 120 is then disassembled from the bushing 112 via a proximal force, and the tissue clip 120 remains within the body at the target tissue site (e.g., target tissue site 150), as shown in FIG. 6. The tissue clip 120 may be left in place until it is removed via natural processes or later through a separate procedure after the bleeding site has healed.

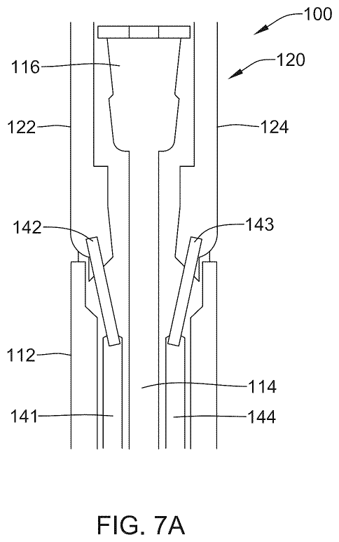

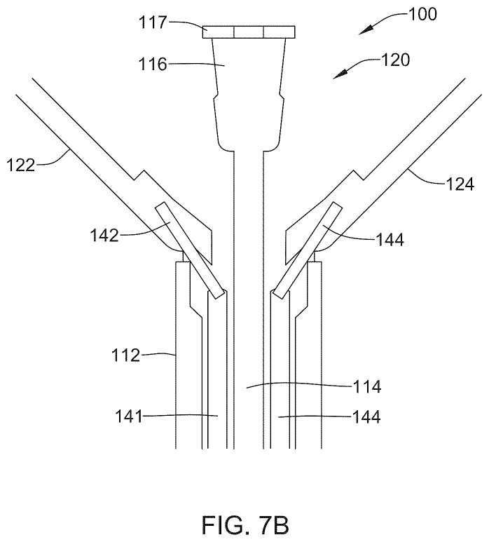

FIGS. 7A to 7B illustrate cross-sectional views of a portion of the example tissue closure device 100. As shown in FIGS. 7A and 7B, the tissue closure device 100 may include an alternative mechanism to radially expand (e.g., open) and radially compress (e.g., close) the tissue closure device 100. As shown in FIGS. 7A and 7B, a first actuation arm 141 and a second actuation arm 144 may be positioned within the bushing 112. The first actuation arm 141 and the second actuation arm 144 may include a first tip 142 and a second tip 143, respectively. The first tip 142 may be coupled to the first arm 122, and the second tip 143 may be coupled to the second arm 124. In use, a user (e.g. a clinician) may utilize an actuation mechanism to advance the actuation arms 141, 144, thereby moving the first tip 142 and the second tip 143, respectively. Distal advancement of the actuation arms 141, 144 causes the first arm 122 and the second arm 124 to radially expand. In some cases, the actuation arms 141, 144 may be retracted proximally, thereby radially compressing the first arm 122 and the second arm 124. In such cases, movement of the control wire 114 is independent from the movement of the actuation arms 141, 144.

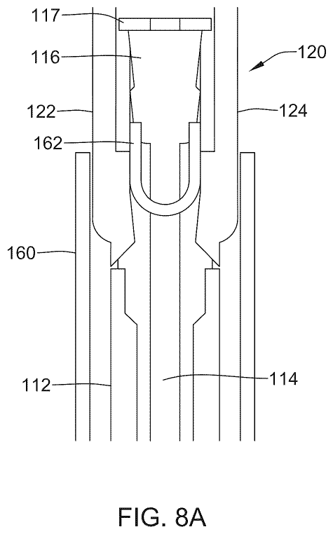

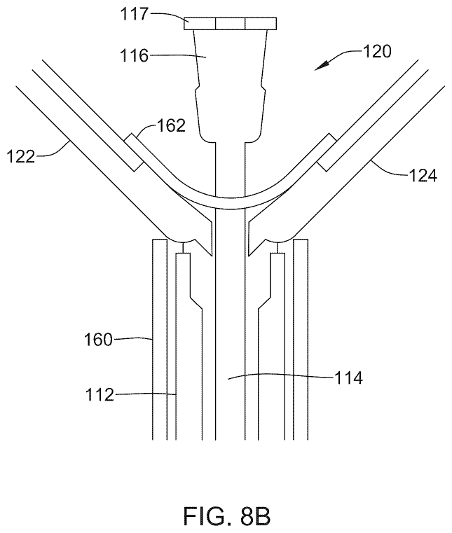

FIGS. 8A to 8B illustrate cross-sectional views of a portion of the example tissue closure device 100. As shown in FIGS. 8A and 8B, the tissue closure device 100 may include an alternative mechanism to radially expand (e.g., open) and radially compress (e.g., close) the tissue closure device 100. As shown in FIGS. 8A and 8B, an outer sleeve 160 may be positioned around an outer diameter of the bushing 112 and may advance distally over the first arm 122 and the second arm 124. When the outer sleeve 160 is advanced over the first arm 122 and the second arm 124, as shown in FIG. 8A, the tissue clip 120 is held in a closed position. When the outer sleeve 160 is retracted proximally, thereby releasing the first arm 122 and the second arm 124, a spring 162 within the tissue clip 120 biases the first arm 122 and the second arm 124 radially outward, thereby moving the tissue clip 120 to an open position.

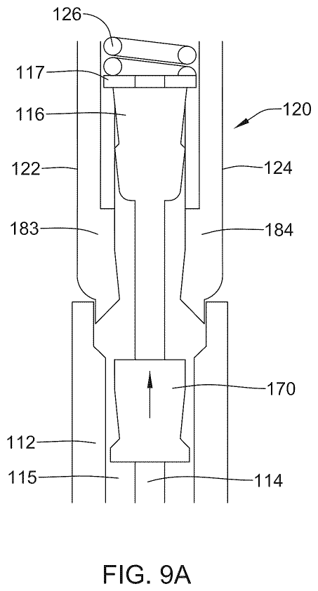

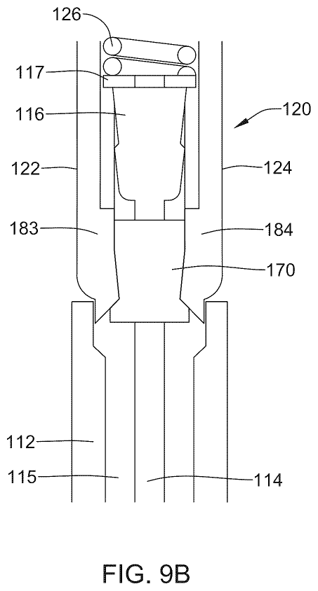

FIGS. 9A to 9B illustrate cross-sectional views of a portion of the example tissue closure device 100. As shown in FIGS. 9A and 9B, the tissue closure device 100 may include an alternative mechanism to radially expand (e.g., open) and radially compress (e.g., close) the tissue closure device 100. As shown in FIGS. 9A and 9B, the tissue closure device 100 may include a locking component 170 that may be advanced distally through the lumen 115 of the bushing 112 and engaging with the ledges 183, 184. The ledges 183, 184 may be configured to receive the locking component 170 and hold the cam member 116 in place when the control wire 114 is removed from the clip 120, thereby locking the first arm 122 and the second arm 124 in the closed position 130, and preventing the cam member 116 from being removed from the clip 120.

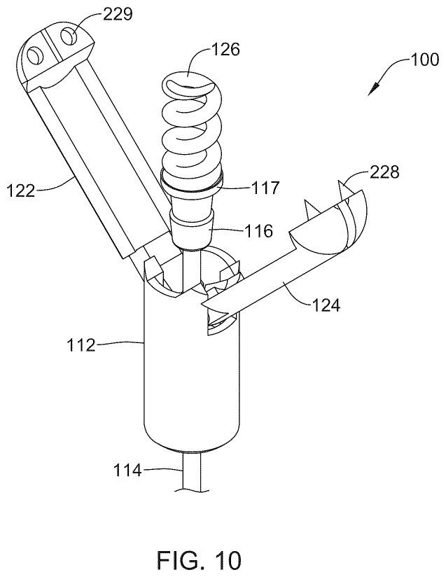

FIG. 10 illustrates the example tissue closure device 100 including alternative tissue grasping members. As shown in FIG. 10, a first tissue grasping member 229 may include one or more recesses which may be configured to receive one or more pyramid points of a second tissue grasping member 228.

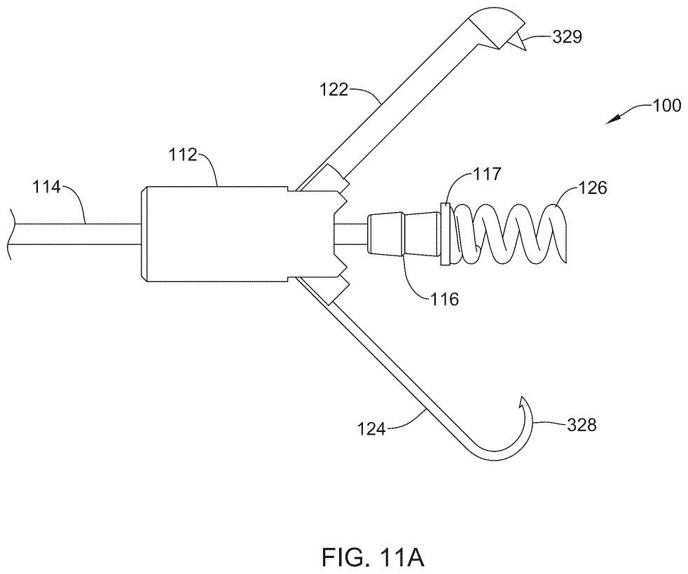

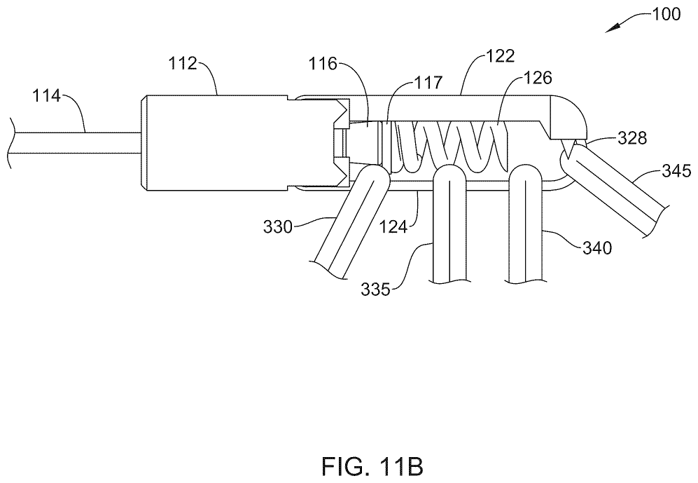

FIGS. 11A to 11B illustrate the example tissue closure device 100 including alternative tissue grasping members. As shown in FIGS. 11A and 11B, the first arm 122 may include a first tissue grasping member 329 which may include one or more pyramid points. In some cases, the second arm 124 may include a second tissue grasping member 328 which may include a hook. In the example of FIGS. 11A and 11B, the second arm 124 may include a smooth, rod like surface configured to allow the second arm 124 to grasp and hold onto multiple sections of target tissue, such as, for example, target tissues 330, 335, 340, 345.

The materials that can be used for the various components of the tissue closure device 10 and the various other medical devices disclosed herein may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the tissue closure device 10 and the various other medical devices disclosed herein may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the tissue closure device 10 and the various other medical devices disclosed herein in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the tissue closure device 10 and the various other medical devices disclosed herein to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A tissue closure device comprising:
a bushing having a distal end, a proximal end, and a lumen extending therethrough;
a clip including at least a first arm and a second arm, wherein a proximal end region of the clip is configured to be coupled to the distal end of the bushing;
a control wire having a proximal end and a distal end, the control wire configured to extend through the lumen of the bushing; and
a helical member configured to be coupled to the distal end of the control wire;
wherein the control wire is configured to advance the helical member in a distal direction along a longitudinal axis of the tissue closure device, and retract the helical member in a proximal direction along the longitudinal axis of the tissue closure device; and
wherein one of the first arm and the second arm includes a tissue grasping member.

2. The tissue closure device of claim 1, wherein the tissue grasping member is a hook.

3. The tissue closure device of claim 1, wherein the helical member is coupled to the control wire via a cam member.

4. The tissue closure device of claim 3, wherein when the control wire is retracted in the proximal direction, the cam member engages with a proximal end of the first arm and a proximal end of the second arm, thereby causing the first arm and the second arm to move toward one another to a closed position.

5. The tissue closure device of claim 4, wherein when the control wire is retracted proximally the cam member is positioned within a proximal end of the clip thereby locking the first arm and the second arm in the closed position.

6. The tissue closure device of claim 1, wherein the proximal end of the control wire is coupled to a handle.

7. The tissue closure device of claim 1, wherein when the clip is in an open position, a proximal end of the first arm and a proximal end of the second arm engage with one another to control an opening angle.

8. A method for tissue closure, the method comprising:
inserting a closure device through a natural body lumen to a target site within a body, the closure device comprising:
a bushing having a distal end, a proximal end, and a lumen extending therethrough;
a clip including at least a first arm and a second arm, wherein a proximal end of the clip is configured to be coupled to the distal end of the bushing;
a control wire having a proximal end and a distal end, the control wire configured to extend through the lumen of the bushing; and
a helical member configured to be coupled to the distal end of the control wire;
advancing the control wire distally so that the first arm and the second arm move away from one another, radially with respect to a longitudinal axis of the clipping device, and the helical member engages a first tissue at the target site;
rotating the control wire in a first direction, which rotates the helical member into the tissue; and
retracting the control wire and the helical member in a proximal direction thereby drawing the first tissue proximally into the clip;
wherein when the control wire and helical member are retracted proximally, the first arm and the second arm move toward one another, thereby closing the clip.

9. The method of claim 8, wherein one of the first arm and the second arm includes a tissue grasping member.

10. The method of claim 9, wherein the tissue grasping member is a hook.

11. The method of claim 8, wherein the helical member is coupled to the control wire via a cam member.

12. The method of claim 11, wherein when the control wire is retracted proximally, the cam member engages with a proximal end of the first arm and a proximal end of the second arm, thereby causing the first arm and the second arm to move toward one another to close the clip.

13. The method of claim 8 further comprising, rotating the control wire in a second direction, which rotates the helical member, thereby disengaging the helical member from the first tissue.

14. The method of claim 13, wherein when the control wire is retracted proximally the cam member is positioned within a proximal end of the clip thereby locking the first arm and the second arm in a closed position.

15. The method of claim 8 further comprising, drawing the control wire proximally to sever a connection between the control wire and the clip, thereby permitting the control wire to be withdrawn from the tissue clipping device.

16. The method of claim 8 further comprising, advancing the control wire so that the first arm and the second arm move away from one another, radially with respect to a longitudinal axis of the clipping device, and the helical member engages a second tissue at the target site.

17. The method of claim 16, wherein when the helical member engages the second tissue, the first tissue is secured to a tissue grasping member.

18. A tissue clip comprising:
a bushing having a distal end and a proximal end;
a first arm including a first tissue grasping member positioned adjacent a distal end of the first arm, wherein a proximal end of the first arm coupled to the distal end of the bushing;
a second arm including a second tissue grasping member positioned adjacent a distal end of the second arm, wherein a proximal end of the second arm coupled to the distal end of the bushing; and
a helical member configured to be positioned between the first arm and the second arm, wherein the helical member is configured to advance distally along a longitudinal axis of the tissue clip;
wherein the tissue clip is configured to move from a first, closed position to a second, open position.

19. The tissue clip of claim 18, wherein when the clip is in the second, open position, a proximal end of the first arm and a proximal end of the second arm engage with one another to control an opening angle.

20. The tissue clip of claim 18, wherein one of the first tissue grasping member or the second tissue grasping member includes a hook.
